(19) United States
(12) Patent Application Publication
(10) Pub. No.: US 2024/0301500 A1
(43) Pub. Date: Sep. 12, 2024

(54) GLOBAL POLYGENIC RISK ASSESSMENT FOR BREAST CANCER

(71) Applicant: MYRIAD GENETICS, INC., Salt Lake City, UT (US)

(72) Inventors: Elisha Hughes, Salt Lake City, UT (US); Alexander Gutin, Salt Lake City, UT (US); Jerry Lanchbury, Salt Lake City, UT (US); Susanne Wagner, Salt Lake City, UT (US)

(73) Assignee: Myriad Genetics, Inc., Salt Lake City, UT (US)

(21) Appl. No.: 18/277,554

(22) PCT Filed: Feb. 24, 2022
(86) PCT No.: PCT/US2022/017697

§ 371 Date: Aug. 16, 2023

(51) Int. Cl. C12Q 1/6886; C12Q 1/6874

(52) U.S. Cl. CPC C12Q 1/6886; C12Q 1/6874; C12Q 2600/106; C12Q 2600/118; C12Q 2600/156

(57) ABSTRACT

Provided herein are methods for assessing a risk of a trait in a subject, by selecting a plurality of ancestry-informative SNP markers based on objective design criteria, measuring a genotype of the subject, obtaining trait-associated SNP markers, and calculating a global polygenic risk score for the risk of the trait in the subject based on the plurality of ancestry-informative SNP markers and the trait-associated SNP markers. The trait can be risk of cancer. Also provided are methods for assessing ancestry of a subject.

### CROSS REFERENCE TO RELATED APPLICATIONS

The present application claims the benefit of priority to U.S. Provisional Application No. 63/153,231 filed Feb. 24, 2021, the entire contents of which are incorporated herein by reference.

### TECHNICAL FIELD

This invention relates to the fields of genetics and medicine. More particularly, this invention relates to methods for assessing and predicting polygenic traits and breast cancer risks for medical use, as well as treating breast cancer.

### BACKGROUND

It is desirable to use polygenomic risk scores to assess the expectation of a clinical trait or condition in a subject such as the risk of a particular disease. Risk scores from genomic data depend on identifying polymorphic loci to be used.

Conventional methods for trait expectation, such as for breast cancer risk, have identified various breast cancer associated genes. However, germline pathogenic variants of breast cancer associated genes introduce complexity that diminishes the accuracy and predictive power of conventional methods. Also, conventional methods may rely on genomic data from a single heritage.

An important drawback in conventional methods for characterizing risk of a trait from genomic data is that baseline data for a trait in one particular population may not accurately predict the same trait in a different population of different heritage. Conventional methods using genomic data from a population drawn from one heritage can overestimate the risk of a particular trait in a different population. Overestimation of risk is a significant drawback, especially for disease traits.

Another drawback of conventional methods for determining traits such as cancer risk include the problem that calculations using genomic data often depend on self-reported heritage information. Errors in self-reported heritage information in genomic data can prevent appropriate determination of cancer risk for a global population.

A significant drawback of conventional methods for determining risk of a trait is a lack of discrimination between low risk and high risk of the trait for different populations. For example, conventional methods for breast cancer risk based on genomic data from one heritage may not be able to distinguish between low risk and high risk for a population of a different heritage. This drawback of conventional methods can confuse prevention and treatment strategies for a disease trait and jeopardize patient outcomes.

Conventional methods for polygenomic risk scores may rely on SNPs discovered through genome-wide association studies (GWAS). However, such SNPs are usually not causal, but may be in linkage disequilibrium (LD) with causal variants. What is need is a set of SNPs for polygenomic risk estimation that will discriminate risk for all heritage groups and populations. It is also desirable to obtain a set of SNP markers for polygenomic risk estimation that do not bias calculations by population, and provide accurate results for all heritage groups and populations.

What is needed is a highly calibrated and accurate method for determining polygenic risk scores for traits such as breast cancer risk to avoid overestimation. There is a need for such methods to be useful for all heritage populations, and regardless of self-reported patient data. An advantageous clinical risk algorithm can improve medical care and patient treatment.

There is an urgent need for methods to assess traits such as breast cancer risk with good discrimination of risk level for all populations regardless of heritage. There is a need for methods that can be efficiently brought to the point of medical care.

### BRIEF SUMMARY

This invention provides methods for determining polygenic traits, such as risks for breast cancer. The methods of this invention can be used in medicine, as well as for treating diseases for which risk is identified and/or assessed.

In some aspects, methods of this invention may provide superior prediction of clinical risk in breast cancer patients. The methods of this invention can provide polygenic risk prediction for breast cancer which can be applied globally to all patients of all heritage groups.

A global polygenomic risk score of this invention can be used to assess the expectation of a clinical trait or condition such as cancer.

Aspects of this invention can characterize an individual's risk of a trait from genomic data obtained for the trait in one particular heritage or population, where the individual may be of a different heritage or population. Embodiments of this invention can provide a global polygenomic risk score for a trait in an individual using genomic data from a population drawn from a different heritage than for the individual, without overestimating the risk of the trait in the individual.

In further aspects, this invention contemplates accurately determining a trait such as cancer risk using genomic data of individuals who self-report heritage information. A global polygenic risk score of this invention can be used to accurately determine cancer risk for a global population, regardless of any errors in self-reported heritage information.

In additional aspects, this invention provides methods for determining risk of a trait with sufficient discrimination between low risk and high risk of the trait for different populations.

In some embodiments, this invention includes methods for breast cancer risk based on a global polygenic risk score that can distinguish between low risk and high risk for a population or individual of any heritage. The methods of this disclosure can provide prevention and treatment strategies for a disease trait and improve patient outcomes.

In further embodiments, this invention provides highly calibrated and accurate methods for determining global polygenic risk scores for traits such as breast cancer risk which avoid overestimation. The methods of this disclosure can be useful for all heritage populations, regardless of self-reported patient data, and can improve medical care and patient treatment.

In additional embodiments, this invention provides methods for assessing traits such as breast cancer risk with enhanced discrimination of risk level for all populations, regardless of heritage. Methods of this disclosure can be efficiently brought to the point of medical care.

Methods of this disclosure further contemplate using various trait risk markers, which may be single nucleotide polymorphisms (SNP). The SNPs of this disclosure may be associated with breast cancer risk in one or more different heritage groups. Combinations of SNPs can be used to provide a global polygenic risk score (gPRS), which can stratify unaffected patients for breast cancer risk, irrespective of the presence or absence of a family history of the disease.

Aspects of this invention provide methods for polygenomic risk scoring that rely on a unique set of SNPs discovered through designated criteria. This unique set of SNPs for polygenomic risk estimation can discriminate risk for all heritage groups and populations. The set of SNP markers for polygenomic risk estimation disclosed herein provide accurate results for all heritage groups and populations, substantially without bias toward any population or heritage group.

Additional classes of markers or elements can include age, family history, breast density, and hormone exposure.

In certain aspects, the clinical utility of this invention may include superior prediction of clinical risk for breast cancer patients of all ancestries.

A global polygenic score obtained by the methods of this invention can provide surprisingly increased accuracy in determining breast cancer risks.

Methods of this invention can provide surprisingly accurate determination of polygenic traits and risks by assessing and including contributions of a wide range of markers for different ancestries.

Embodiments of this invention contemplate determining the levels of polygenic traits and risks in the form of a score based on various genomic risk loci. The genomic risk loci can be discretely identified and defined, so that accurate determination can be done by genotyping subjects.

In certain aspects, the genomic risk loci can include genomic risk markers for breast cancer, which are combined with additional risk markers that can be specifically breast cancer-informative.

### Embodiments of this Invention Include

A method for assessing ancestry of a subject, the method comprising:
selecting a plurality of ancestry-informative SNP markers based on the criteria:
   the SNP markers substantially cover the entirety of the human genome;
   the SNP markers each have at least 1% genomic frequency; and
   the SNP markers have different frequencies in different heritage populations;
   measuring a genotype of the subject; and
calculating a fractional heritage in the genotype of the subject for each of the different heritage populations based on the plurality of ancestry-informative SNP markers. The ancestry-informative SNP markers may have different frequencies in three or more different heritage populations, such as African, European, and East Asian heritage populations.

The plurality of ancestry-informative SNP markers can be from 10 to 50,000 SNP markers, or from 10 to 56 SNP markers.

A method for assessing a risk of a trait in a subject, the method comprising:
selecting a plurality of ancestry-informative SNP markers based on the criteria:
   the SNP markers substantially cover the entirety of the human genome;
   the SNP markers each have at least 1% genomic frequency; and
   the SNP markers have different frequencies in different heritage populations; measuring a genotype of the subject;
obtaining trait-associated SNP markers; and
calculating a global polygenic risk score for the risk of the trait in the subject based on the plurality of ancestry-informative SNP markers and the trait-associated SNP markers. The calculating the global polygenic risk score for the risk of the trait in the subject can be done with additional clinical variables of the subject, such as age, personal medical history, and family medical history of the subject. The trait can be a risk of a disease in the subject, such as cancer.

The plurality of ancestry-informative SNP markers can be from 10 to 50,000 SNP markers, or from 10 to 56 SNP markers. The trait-associated SNP markers can be a plurality of cancer-associated SNP markers. The trait-associated SNP markers may be a plurality of from 10 to 50,000 breast cancer-associated SNP markers, or from 10 to 93 breast cancer-associated SNP markers.

The method above, wherein the calculating a global polygenic risk score for the risk of the trait in the subject can be done with training clinical data of a reference group, or with validating clinical data of a reference group. The genotype of the subject can be measured by NGS, or with a sequencing chip.

The method above, wherein the plurality of ancestry-informative SNP markers can determine a fractional heritage in the genotype of the subject for each of three or more different heritage populations, such as African, European, and East Asian heritage populations.

The method above, wherein the global polygenic risk score for the risk of the trait in the subject may be accurate for subjects in three or more different heritage populations, even when the heritage populations are self-reported, such as African, European, and East Asian heritage populations.

The method above, wherein the global polygenic risk score for the risk of the trait in the subject can be calibrated for subjects in three or more different heritage populations so that the risk of the trait is not overestimated in any heritage population, such as African, European, and East Asian heritage populations.

The method above, wherein the global polygenic risk score for the risk of the trait in the subject can discriminate between low risk and high risk for subjects in three or more different heritage populations, such as African, European, and East Asian heritage populations.

The methods above, wherein the trait is a risk of a disease in the subject, such as cancer.

The method above, wherein the calculating a global polygenic risk score can comprise using clinical cohorts of women of African self-reported ancestry, East Asian self-reported ancestry, and European self-reported ancestry.

The method above, wherein the calculating a global polygenic risk score can comprise using the sum of ancestry specific polygenic risk scores weighted according to fractional ancestral composition.

The method above, wherein the global polygenic risk score may be strongly associated with breast cancer in a reference cohort and in sub-cohorts defined by self-reported ancestry.

The method above, wherein the global polygenic risk score can be combined with clinical and/or biological risk factors for accurate risk stratification for all women of all ancestries.

The method above, wherein the calculating a global polygenic risk score can comprise a linear combination of risk alleles according to Equation III.

$$Polygenic Risk Score = {b}_{1} ( {x}_{1} - {u}_{1} ) + {b}_{2} ( {x}_{2} - {u}_{2} ) + … + {b}_{N} ( {x}_{N} - {u}_{N} ) ; \qquad Equation III$$

where N is the total number of SNPs selected;
the coefficient bₖ is the per-allele log OR for trait association of the kth SNP estimated from a development cohort;
xₖ is the number of alleles of the kth SNP carried by an individual patient which is 0, 1 or 2;
and uₖ is the average number of alleles of the kth SNP reported for individuals included in large general population studies.

Embodiments of this invention further contemplate methods for treating a disease in a subject in need thereof, the method comprising:
selecting a plurality of ancestry-informative SNP markers based on the criteria:
   the SNP markers substantially cover the entirety of the human genome;
   the SNP markers each have at least 1% genomic frequency; and
   the SNP markers have different frequencies in different heritage populations;
measuring a genotype of the subject;
obtaining disease-associated SNP markers; and
calculating a global polygenic risk score for the risk of the disease in the subject based on the plurality of ancestry-informative SNP markers and the disease-associated SNP markers, wherein the score indicates a need for treating the subject; and
administering to the subject a therapy for the disease. The calculating the global polygenic risk score may be done with additional variables for age, personal medical history, and family medical history. The disease may be cancer. The therapy can be a cancer therapy selected from one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound. The disease may be breast cancer. The therapy can be a breast cancer therapy.

This invention includes methods for diagnosing or prognosing a subject having a disease, the method comprising:
selecting a plurality of ancestry-informative SNP markers based on the criteria:
   the SNP markers substantially cover the entirety of the human genome;
   the SNP markers each have at least 1% genomic frequency; and
   the SNP markers have different frequencies in different heritage populations;
measuring a genotype of the subject;
obtaining disease-associated SNP markers; and
calculating a global polygenic risk score for the risk of the disease in the subject based on the plurality of ancestry-informative SNP markers and the disease-associated SNP markers, wherein the score indicates a diagnosis or prognosis for the subject. The disease may be cancer.

This invention includes methods for generating data for assessing a trait in a subject, the method comprising:
selecting a plurality of ancestry-informative SNP markers based on the criteria:
   the SNP markers substantially cover the entirety of the human genome;
   the SNP markers each have at least 1% genomic frequency; and
   the SNP markers have different frequencies in different heritage populations;
measuring a genotype of the subject;
measuring trait-associated SNP markers in the genotype of the subject. The method may further comprise determining additional clinical variables of the subject, such as age, personal medical history, and family medical history of the subject. The trait can be a risk of a disease in the subject, such as cancer. The plurality of ancestry-informative SNP markers are from 10 to 50,000 SNP markers, or from 10 to 56 SNP markers. The trait-associated SNP markers can be a plurality of cancer-associated SNP markers. The trait-associated SNP markers may be a plurality of from 10 to 50,000 breast cancer associated SNP markers, or from 10 to 93 breast cancer associated SNP markers.

This invention further includes systems for assessing risk of a disease in a subject, the system comprising:
a processor for receiving a genotype of the subject;
one or more processors for carrying out the steps:
   calculating a global polygenic risk score for risk of the disease in the subject based on a plurality of ancestry-informative SNP markers, a plurality of disease-associated SNP markers of the genotype, and additional variables for age, personal medical history, and family medical history; and
a display for displaying and/or reporting the risk score. The disease may be cancer.

Additional embodiments include non-transitory machine-readable storage media having stored therein instructions for execution by a processor which cause the processor to perform the steps of a method for assessing risk of a disease in a subject, the method comprising:
receiving a genotype of the subject;
calculating a global polygenic risk score for risk of the disease in the subject based on a plurality of ancestry-informative SNP markers, a plurality of disease-associated SNP markers of the genotype, and additional variables for age, personal medical history, and family medical history; and
sending to a processor output for displaying and/or reporting the risk score. The disease may be cancer.

### BRIEF DESCRIPTION OF THE DRAWINGS

FIG. 1 shows an illustration of ancestry in terms of contributions from different continents.

FIG. 2 shows an illustration of a distribution of genotypes based on ancestry for Hispanic, White/non-Hispanic, Black/African, and Asian genotypes.

FIG. 3 shows an illustration of an embodiment of this invention in which the distribution of Global Polygenic Risk Scores for breast cancer risk was centered about zero for patients of all ancestry genotypes indicating removal of ancestry-derived bias from risk estimation.

FIG. 4 shows an illustration of an embodiment of this invention in which the distribution of Global Polygenic Risk Scores for breast cancer risk was centered about zero for Hispanic patients who do not carry a 6q25 SNP (rs140068132).

FIG. 5 shows a comparison of a ancestry-specific differences in distributions of polygenic risk scores based on different breast cancer allele frequencies.

FIG. 6 shows an illustration of historical breast cancer rates by ancestry.

### DETAILED DESCRIPTION OF THE DISCLOSURE

This invention includes methods for determining a global polygenic risk score which can be predictive for a trait in a subject.

A global polygenic risk score can be predictive for risk assessment for breast cancer.

In some aspects, this invention provides methods for global polygenic risk prediction with surprisingly increased accuracy of risk assessment for a trait in a subject.

Embodiments of this invention further provide reliable breast cancer risk associations applicable to all populations of all ancestries.

This disclosure provides various methods for clinical risk management, risk magnitude assessment, as well as global polygenic risk scores, and non-clinical trait prediction. Methods of this invention can provide predictive ability that is surprisingly accurate for populations of all ancestries.

Aspects of this disclosure include genotyping a subject using various markers associated with a disease and combining the genotypes in the form of a global polygenic risk score to predict risk of a trait, such as a clinical condition or an extent of manifestation of a biological trait.

In further embodiments, a plurality of trait risk markers can be used to provide a global polygenic risk prediction for the trait.

The plurality of trait risk markers may include various disease-associated gene markers.

In some embodiments, the plurality of trait risk markers may include from 1-1,000,000 SNP markers.

In certain embodiments, the plurality of trait risk markers may include from 1-10,000 SNP markers, or from 1-1000 SNP markers, or from 1-100 SNP markers. A plurality of trait risk markers may be from 1-1000 breast cancer SNP markers, or from cancer 1-500 breast cancer SNP markers, or from 1-100 breast cancer SNP markers.

In certain embodiments, the plurality of trait risk markers may include 56 SNP markers to 149 SNP markers.

This invention provides methods for determining polygenic traits, such as risks for disease including breast cancer. The methods of this invention can be used for treating diseases for which risk is determined through polygenomic scoring.

In some embodiments, methods of this invention may provide superior prediction of clinical risk in breast cancer patients. The methods of this invention can provide global polygenic risk prediction for disease such as breast cancer which can be applied globally to all patients of all heritage groups.

A global polygenomic risk score of this invention can be used to assess the expectation of a clinical trait or condition such as cancer in a subject.

In certain embodiments, this invention can calculate an individual's risk of a trait from genomic data obtained for the trait in one particular heritage group or population, where the individual may be of a different heritage group or population. Embodiments of this invention can therefore provide a global polygenomic risk score for a trait in an individual using genomic data from a population drawn from a different heritage than the heritage to which the individual belongs or has self-identified, without overestimating the risk of the trait in the individual.

In further embodiments, this invention contemplates accurately determining a trait such as cancer risk using genomic data of individuals who self-report heritage information. A global polygenic risk score of this invention can be used to accurately determine cancer risk for a subject of any heritage, regardless of any errors in self-reported heritage information.

In additional embodiments, this invention provides methods for determining risk of a trait in an individual with sufficient discrimination between low risk and high risk for the trait, regardless of the heritage group or population to which the subject belongs or has self-identified.

In some embodiments, this invention includes methods for breast cancer risk based on a global polygenic risk score that can distinguish between low risk and high risk, surprisingly for an individual of any heritage.

The methods of this disclosure can provide prevention and treatment strategies for a disease trait to improve patient outcomes.

In further embodiments, this invention can provide global polygenic risk scores that are highly calibrated and accurate. The global polygenic risk scores can be used in methods for determining traits such as breast cancer risk in subjects which avoid overestimation. The methods of this disclosure can be useful for all heritage groups and/or populations, regardless of the use of self-reported patient data, and can improve medical care and patient treatment.

In additional embodiments, this invention provides methods for assessing traits such as breast cancer risk with enhanced discrimination of risk level for all populations, regardless of heritage. Methods of this disclosure can be efficiently brought to the point of medical care.

Methods of this disclosure further contemplate using various trait risk markers, which may be single nucleotide polymorphisms (SNP). The SNPs of this disclosure may be associated with breast cancer risk in one or more different heritage groups. Combinations of SNPs can be used to provide a global polygenic risk score (gPRS), which can stratify unaffected patients for breast cancer risk, irrespective of the presence or absence of a family history of the disease.

Additional classes of markers or elements can include age, family history, breast density, and hormone exposure.

In certain aspects, the clinical utility of this invention may include superior prediction of clinical risk for breast cancer patients of all ancestries.

A global polygenic score obtained by the methods of this invention can provide surprisingly increased accuracy in determining breast cancer risks.

Methods of this invention can provide surprisingly accurate determination of polygenic traits and risks by assessing and including contributions of a wide range of markers for different ancestries.

Embodiments of this invention contemplate determining the levels of polygenic traits and risks in the form of a score based on various genomic risk loci. The genomic risk loci can be discretely identified and defined, so that accurate determination can be done by genotyping subjects.

In certain aspects, the genomic risk loci can include genomic risk markers for breast cancer, which are combined with additional risk markers that can be specifically breast cancer-informative.

In additional embodiments, the plurality of trait risk markers may include from 1-100 family history elements, or from 1-20 family history elements, or from 1-10 family history elements.

Embodiments of this invention may include a plurality of trait risk markers such as from 1-100 clinical elements, or from 1-20 clinical elements, or from 1-10 clinical elements.

Embodiments herein can provide improved global polygenic risk prediction for breast cancer.

Comprehensive risk assessment combining a polygenic SNP scoring method with other risk factors and elements can improve the accuracy of risk estimates and facilitate decision-making for women with pathogenic variants in moderately penetrant genes.

In further aspects, a polygenic risk score of this invention may be surprisingly more accurate for breast cancer than using conventional methods.

In certain aspects, an association between the global polygenic risk scores and breast cancer may be evaluated by fixed stratification methods. The fixed stratification may be adjusted for age and family history, among other variables and elements.

Embodiments of this invention can provide women an estimated lifetime risk for breast cancer with increased accuracy. Such risk estimation is useful to inform decisions based on a threshold for more aggressive screening, including consideration of breast magnetic resonance imaging (MRI).

In some aspects, disclosed herein are methods that can utilize breast cancer SNP markers to provide a global polygenic risk score for breast cancer.

Some examples of breast cancer risk markers are given in: Prediction of breast cancer risk based on profiling with common genetic variants, Mavaddat et al., J Natl Cancer Inst., 2015 Apr. 8, Vol. 107(5), djv036.

Some examples of breast cancer risk markers are given in: Michailidou et al., Genome-wide association analysis of more than 120,000 individuals identifies 15 new susceptibility loci for breast cancer, Nat Genet., 2015, Vol. 47, pp. 373.

Some examples of breast cancer risk markers are given in Characterizing Genetic Susceptibility to Breast Cancer in Women of African Ancestry, Feng et al., Cancer Epidemiol Biomarkers Prev., 2017 July, Vol. 26(7), pp. 1016-1026.

Some examples of breast cancer risk markers are given in Rainville, I. et al., Breast Cancer Research and Treatment, 2020, Vol. 180, pp. 503-509.

Some examples of breast cancer risk markers are given in Early Diagnosis of Breast Cancer, Wang et al., Sensors (Basel), 2017 July, Vol. 17(7), p. 1572.

Some examples of genetic modifiers for breast cancer risk are given in Muranen T A, et al., Genetics in Medicine, 2017, Vol. 19(5), pp. 599-603.

Some examples of risk scores for breast cancer are given in Kuchenbaecker K, et al., J Natl Cancer Inst., 2017, Vol. 109(7), djw302.

Some examples for cancer risk are given in: Perencevich M, et al., Gastroenterology & Hepatology, 2011, Vol. 7(6), pp. 420-423.

Some examples for gene analysis are given in: Lek et al., Nature, 2016, Vol. 536.7616, pp. 285.

### Ancestry-Informative SNPs

In general, a polygenic determination of a trait in a subject can be done with a set of polygenic SNP markers. In some embodiments, the trait can be ancestry.

Aspects of this invention provide advantages in characterizing the genotype of a subject according to ancestry.

In certain aspects, methods of this disclosure can use SNPs associated with one or more different heritage groups. Combinations of SNPs can be used for assessing the ancestry of a subject. A genotype of a subject can be determined based on fractional ancestry of one or more different heritage groups.

Embodiments of this invention provide methods for assessing ancestry of a subject by selecting a plurality of ancestry-informative SNP markers. The ancestry-informative SNP markers can be based on one or more criteria such as the ability to substantially cover the entirety of the human genome, having at least 1% genomic frequency, and having different frequencies in different heritage populations. By obtaining the genotype of a subject, a fractional heritage in the genotype of the subject can be calculated for each of the different heritage populations based on the plurality of ancestry-informative SNP markers.

In some embodiments, the ancestry-informative SNP markers can have different frequencies in three or more different heritage populations, such as in African, European, and East Asian heritage populations. A plurality of from 10 to 50,000 ancestry-informative SNP markers can be used.

In some embodiments, the plurality of ancestry-informative SNP markers may include from 1-1,000,000 SNP markers.

In certain embodiments of this invention, a plurality of 10 to 56 ancestry-informative SNP markers can been used.

Methods of this disclosure can combine the use of the ancestry-informative SNP markers with additional SNP markers that may be associated with a biological trait. Combinations of SNPs can be used to provide a global polygenic risk score (gPRS), which can stratify subjects for risk of the trait regardless of heritage. A global polygenic risk score can inherently incorporate genomic information based on fractional ancestry.

Aspects of this invention provide methods for global polygenomic risk scoring that rely on a unique set of SNPs discovered through design criteria. This unique set of SNPs for global polygenomic risk estimation can discriminate risk for all heritage groups and populations. The unique set of SNP markers for polygenomic risk estimation disclosed herein provide accurate results for all heritage groups and populations, substantially without bias toward any population or heritage group.

In certain aspects, a unique set of ancestry-informative SNP markers was discovered using estimates of individual SNP risk betas in different ancestry groups. In some embodiments, for each ancestry, individual SNP risk betas were determined from known values, from data obtained in myRisk patients, and through meta-analysis of combined data of the foregoing. Estimates of individual SNP risk betas in different ancestry groups can be used to determine a unique set of SNP markers that can provide a global polygenic risk score for risk of a trait, such as cancer risk, and which can stratify unaffected patients for risk irrespective of ancestry.

For example, in some embodiments, African SNP risk betas can be determined from 1,000 or more, or from 5,000 or more, or from 10,000 or more myRisk measurements of patients of self-reported African ancestry. About seventy Asian SNP risk betas can be determined from Shu et al., Nat Commun., 2020, Vol. 11, pp. 1217-1226. Hispanic SNP risk betas can be determined from 1,000 or more, or from 5,000 or more, or from 10,000 or more Hispanic myRisk measurements of patients of self-reported Hispanic ancestry.

Embodiments of this invention can provide a global polygenic risk score for a trait that can be clinically validated for all women of all heritage groups and populations.

A global polygenic risk score of this disclosure can provide meaningful risk discrimination of a trait for all women of all heritage groups and populations.

A global polygenic risk score of this disclosure can provide a statistical distribution of scores for a trait for a population, where the scores can be centered at zero with no bias for any ancestry-specific subpopulation.

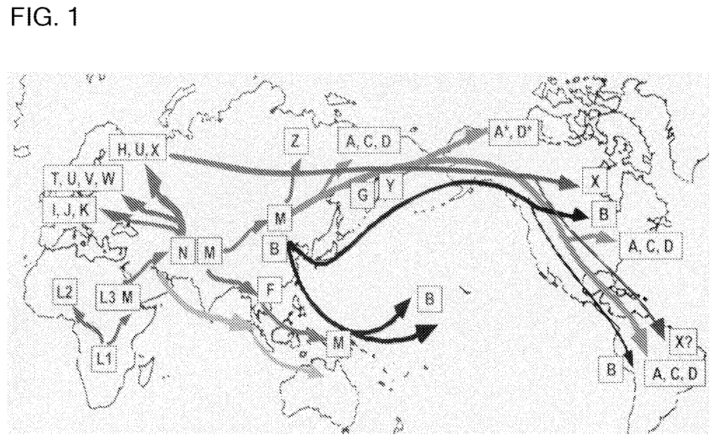

FIG. 1 shows an illustration of ancestry in terms of contributions from different continents.

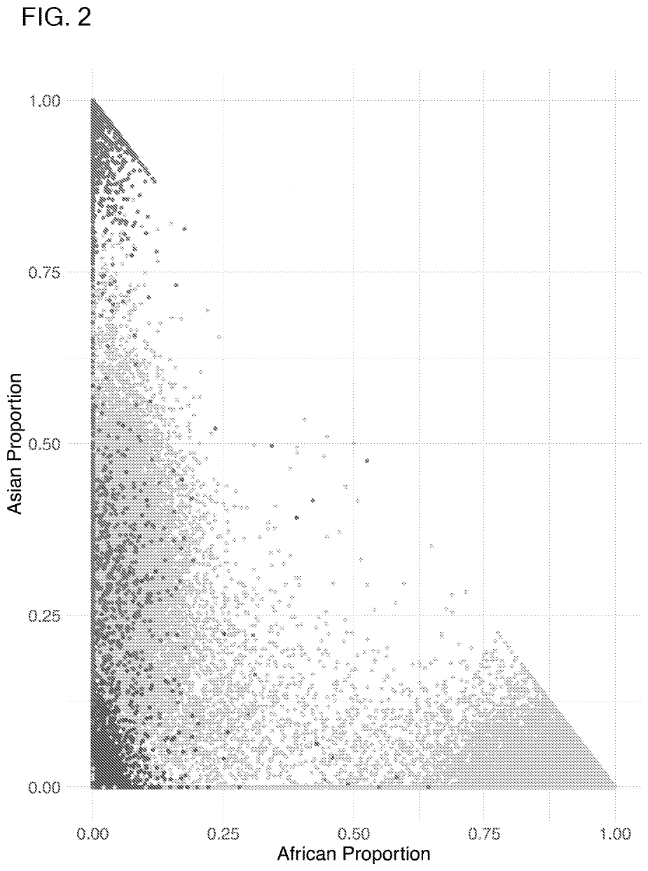

FIG. 2 shows an illustration of a distribution of genotypes based on ancestry for Hispanic, White/non-Hispanic, Black/African, and Asian genotypes.

A unique set of ancestry-informative SNPs for global polygenomic risk estimation can be obtained by characterizing the ancestry of a subject in terms of contributions from different continents.

In further embodiments, a unique set of ancestry-informative SNP markers can be obtained by design criteria to distinguish between three continental ancestries: African, East Asian and European.

In further embodiments, a unique set of 56 ancestry-informative SNP markers can be obtained by design criteria to distinguish between three or more continental ancestries.

Ancestry-informative SNPs of this disclosure include those in Table 1.

TABLE 1
Ancestry-informative SNPs

| | | |
|---|---|---|
| No. | SNP | Chromosome |

| | |
|---|---|
| 1 | rs10041728 |
| 2 | rs10152250 |
| 3 | rs1063 |
| 4 | rs10853040 |
| 5 | rs1089605 |
| 6 | rs11150606 |
| 7 | rs11778591 |
| 8 | rs1229984 |
| 9 | rs12913832 |
| 10 | rs1314014 |
| 11 | rs13236072 |
| 12 | rs1369290 |
| 13 | rs1641127 |
| 14 | rs16891982 |
| 15 | rs16932430 |
| 16 | rs17093005 |
| 17 | rs172447 |
| 18 | rs17614025 |
| 19 | rs1811510 |
| 20 | rs1834640 |
| 21 | rs186332 |
| 22 | rs1866694 |
| 23 | rs1871534 |
| 24 | rs2196051 |
| 25 | rs2268421 |
| 26 | rs2384319 |
| 27 | rs2416791 |
| 28 | rs2789823 |
| 29 | rs2814778 |
| 30 | rs289816 |
| 31 | rs344454 |
| 32 | rs3768641 |
| 33 | rs3799027 |
| 34 | rs3827760 |
| 35 | rs3905643 |
| 36 | rs4918664 |
| 37 | rs58827274 |
| 38 | rs6046748 |
| 39 | rs61691136 |
| 40 | rs6456591 |
| 41 | rs67364864 |
| 42 | rs6754311 |
| 43 | rs67875794 |
| 44 | rs6869589 |
| 45 | rs6930377 |
| 46 | rs7135361 |
| 47 | rs7201030 |
| 48 | rs7611642 |
| 49 | rs7927064 |
| 50 | rs79525262 |
| 51 | rs7959696 |
| 52 | rs798789 |
| 53 | rs8097206 |
| 54 | rs9411013 |
| 55 | rs9522149 |
| 56 | rs953035 |

### Global Polygenomic Risk Estimation for Cancer

Embodiments of this invention further contemplate combining a unique set of ancestry-informative SNP markers with additional SNP markers associated with a trait such as risk of cancer.

In some embodiments, methods of this invention can combine the use of ancestry-informative SNP markers with additional SNP markers that may be associated with cancer risk in one or more different heritage groups. Combinations of such SNPs can be used to provide a global polygenic risk score (gPRS) for cancer risk, which can stratify unaffected patients for cancer risk, irrespective of ancestry and the presence or absence of a family history of the disease.

In further embodiments, methods of this invention can combine the use of ancestry-informative SNP markers with additional SNP markers that may be associated with breast cancer risk in women of one or more different heritage groups. Combinations of such SNPs can be used to provide a global polygenic risk score (gPRS) for breast cancer risk, which can stratify unaffected women for breast cancer risk, irrespective of ancestry and the presence or absence of a family history of the disease.

In some aspects, a global polygenomic risk estimation may characterize the risk of cancer in a subject regardless of the subject's genetic ancestry by using a combination of ancestry-informative SNP markers and cancer-associated SNPs. The cancer-associated SNPs may be derived from one or more different heritage groups or populations.

In some aspects, a global polygenomic risk estimation may characterize the risk of breast cancer in a woman regardless of genetic ancestry by using a combination of ancestry-informative SNP markers and breast cancer-associated SNPs. The breast cancer-associated SNPs may be derived from one or more different heritage groups or populations.

In some embodiments, a global polygenomic risk score may characterize the risk of breast cancer in a woman regardless of genetic ancestry by using a combination of from 10 to 56 ancestry-informative SNP markers and from 10 to 93 breast cancer-associated SNPs. The breast cancer-associated SNPs can include up to 92 European breast cancer-associated SNPs and one Hispanic breast cancer SNP 6q25 (rs140068132).

In certain embodiments, a global polygenomic risk score may characterize the risk of breast cancer in a woman regardless of genetic ancestry by using a combination of 56 ancestry-informative SNP markers and 93 breast cancer-associated SNPs comprised of 92 European breast cancer-associated SNPs and one Hispanic breast cancer SNP 6q25 (rs140068132).

A global polygenomic risk score of this invention can achieve a high level of accuracy for all women of all heritage groups and populations in terms of surprisingly high risk discrimination and superior accuracy of calibration.

Breast cancer-associated SNPs of this disclosure include those in Table 2.

TABLE 2
Breast cancer-associated SNPs

| No. | SNP | Chromosome |
|---|---|---|
| 1 | rs10069690 | |
| 2 | rs1011970 | |
| 3 | rs1045485 | |
| 4 | rs10472076 | |
| 5 | rs1053338 | |
| 6 | rs10759243 | |
| 7 | rs10771399 | |
| 8 | rs10941679 | |
| 9 | rs10995190 | |
| 10 | rs11075995 | |
| 11 | rs11199914 | |
| 12 | rs11242675 | |
| 13 | rs11249433 | |
| 14 | rs11552449 | |
| 15 | rs11571833 | |
| 16 | rs11627032 | |
| 17 | rs11780156 | |
| 18 | rs11814448 | |
| 19 | rs11820646 | |
| 20 | rs12405132 | |
| 21 | rs12422552 | |
| 22 | rs12493607 | |
| 23 | rs12662670 | |
| 24 | rs12710696 | |
| 25 | rs1292011 | |
| 26 | rs13162653 | |
| 27 | rs132390 | |
| 28 | rs13267382 | |
| 29 | rs13281615 | |
| 30 | rs13329835 | |
| 31 | rs13365225 | |
| 32 | rs13387042 | |
| 33 | rs1353747 | |
| 34 | rs140068132 | 6q25 |
| 35 | rs1432679 | |
| 36 | rs1436904 | |
| 37 | rs1550623 | |
| 38 | rs16857609 | |
| 39 | rs17356907 | |
| 40 | rs17529111 | |
| 41 | rs17817449 | |
| 42 | rs17879961 | |
| 43 | rs2012709 | |
| 44 | rs2016394 | |
| 45 | rs204247 | |
| 46 | rs2046210 | |
| 47 | rs2236007 | |
| 48 | rs2363956 | |
| 49 | rs2380205 | |
| 50 | rs2588809 | |
| 51 | rs2736108 | |
| 52 | rs2823093 | |
| 53 | rs2943559 | |
| 54 | rs2981579 | |
| 55 | rs3760982 | |
| 56 | rs3803662 | |
| 57 | rs3817198 | |
| 58 | rs3903072 | |
| 59 | rs4245739 | |
| 60 | rs4593472 | |
| 61 | rs4808801 | |
| 62 | rs4849887 | |
| 63 | rs4973768 | |
| 64 | rs527616 | |
| 65 | rs554219 | |
| 66 | rs6001930 | |
| 67 | rs616488 | |
| 68 | rs62070644 | |
| 69 | rs6472903 | |
| 70 | rs6504950 | |
| 71 | rs6507583 | |
| 72 | rs6678914 | |
| 73 | rs6762644 | |
| 74 | rs6796502 | |
| 75 | rs6828523 | |
| 76 | rs6964587 | |
| 77 | rs704010 | |
| 78 | rs7072776 | |
| 79 | rs720475 | |
| 80 | rs72755295 | |
| 81 | rs745570 | |
| 82 | rs75915166 | |
| 83 | rs7707921 | |
| 84 | rs7726159 | |
| 85 | rs78540526 | |
| 86 | rs7904519 | |
| 87 | rs8170 | |
| 88 | rs865686 | |
| 89 | rs889312 | |
| 90 | rs941764 | |
| 91 | rs9693444 | |
| 92 | rs9790517 | |
| 93 | rs999737 | |

### Polygenic Risk Scores

In another example, a polygenic estimation of breast cancer risk can be made using an 86-SNP polygenic risk score. A 86-SNP Polygenic Risk Score can provide association with risk of breast cancer development in women carrying pathogenic variants in low to moderately penetrant genes such as ATM, CHEK2, and PALB2. The absolute risks of breast cancer to age 80 can be calculated to illustrate the potential clinical utility of polygenic stratification in women with pathogenic variants in BRCA1/2, ATM, CHEK2, and PALB2.

A polygenic risk score can be defined as a linear combination of centered risk alleles according to Equation III.

$$Polygenic Risk Score = {b}_{1} ( {x}_{1} - {u}_{1} ) + {b}_{2} ( {x}_{2} - {u}_{2} ) + … + {b}_{N} ( {x}_{N} - {u}_{N} ) ; \qquad Equation III$$

where N is the total number of SNPs selected, the coefficient bₖ is the per-allele log OR for breast cancer association of the kth SNP estimated from meta-analysis of literature and the development cohort; xx is the number of alleles of the kth SNP carried by an individual patient (Xₖ=0, 1 or 2); and uₖ is the average number of alleles of the kth SNP reported for individuals included in large general population studies. Passing criteria may restrict the number of missing SNP calls such that the imputation of missing calls by the high or low risk allele(s) does not change the relative risk by more than 10%.

In some aspects, SNP coefficients can be estimated for the polygenic risk score.

In some embodiments, SNP coefficients can be estimated and standard errors for a plurality of pertinent SNPs can be obtained based on a development cohort. These coefficients can be designated {b_devk| k=1, 2, . . . , N_{SNP}}, and standard errors by {σ_devk k=1, 2, . . . , N_{SNP}}, where N_{SNP} is the number of SNPs used. These values can be estimated from a single multivariate logistic regression model with breast cancer status as the dependent variable, and the following independent variables: N_{SNP} numeric variables representing allele counts for each of N_{SNP} SNPs {xₖ| k=1, 2, . . . , N_{SNP}}, age, ancestry, personal cancer history, and family cancer history. Age, ancestry, personal and family cancer history variables may be coded as described above. SNP coefficients can further be estimated by selecting literature-based coefficients {b_litk| k=1, 2, . . . , N_{SNP}}, and standard errors {σ_litk| k=1, 2, . . . , N_{SNP}}. Linkage disequilibrium between SNPs can be accounted for by co-estimating the effects in multivariate regression models, with one model for each gene.

Lastly, polygenic risk score coefficients can be calculated according to {bₖ| k=1, 2, . . . , N_{SNP}} from a meta-analysis of development cohort and literature-based coefficients. Polygenic risk score coefficients may be calculated as weighted averages of development cohort and literature coefficients with weights inversely proportional to squared standard errors. The ratio of squared standard errors can be replaced with the median value.

More specifically, for a plurality of SNPs, and with non-missing σ_litk values, a median ratio can be calculated according to Equation IV.

$$median\_ratio = median of [ {( \frac{{σ\_lit}_{1}}{{σ\_dev}_{1}} )}^{2} , {( \frac{{σ\_lit}_{2}}{{σ\_dev}_{2}} )}^{2} , … , {( \frac{{σ\_lit}_{Nsnp}}{{σ\_dev}_{Nsnp}} )}^{2} ] . \qquad Equation IV$$

where, for each k in 1 through N_{SNP}, bₖ was defined according to Equation V

$${b}_{k} = \frac{median\_ratio \times {b\_dev}_{k} + {b\_lit}_{k}}{1 + median\_ratio} . \qquad Equation V$$

In further aspects, the informativeness of each SNP can be calculated.

The informativeness of a SNP may be a function if its effect size, and its general population allele frequency. For each k in 1 through N_{SNP}, informativeness of the kth SNP can be calculated according to Equation VI.

$$2 \times {b}_{k}^{2} \times \frac{1}{2} {u}_{k} \times ( 1 - \frac{1}{2} {u}_{k} ) . \qquad Equation VI$$

In additional aspects, SNPs may be ordered by informativeness. By designation, b₁ may denote the most informative SNP, b₂ the second most informative SNP, and so on.

Chi-square likelihood ratio test (LRT) statistics can be calculated to evaluate the contribution of each SNP to the polygenic risk score (PRS). For SNPs from linked sets, only the single most informative representative SNP from each gene may be included, leaving N_{SNP} less one for evaluation. For each k in 1 through N_{SNP}, analyses can be made in a development cohort according to the following steps. First, calculate k-SNP PRS scores for all patients according to Equation VII.

$$P R {S}_{k} = {b}_{1} ( {x}_{1} - {u}_{1} ) + {b}_{2} ( {x}_{2} - {u}_{2} ) + … + {b}_{k} ( {x}_{k} - {u}_{k} ) . \qquad Equation VII$$

Secondly, construct a multivariate logistic regression model with breast cancer status as the dependent variable, and independent variables for PRSₖ, age, ancestry, personal cancer history, and family cancer history. Third, record the LRT statistic comparing the full model to the nested model with PRSₖ omitted.

In further aspects, SNPs for a PRS may be selected according to highest likelihood ratio test (LRT) value. All linked SNPs from a gene may be included if the representative SNP was selected for inclusion.

The identity of a plurality of SNPs incorporated into an 86-SNP score embodiment are shown in Table 3. Chromosomal positions are given according to hg19.

TABLE 3
SNPs incorporated into an 86-SNP polygenic risk score

| | | | | 86-SNP |
|---|---|---|---|---|
| MARKER | SOURCE | CHR | POSITION | SCORE |
| rs616488 | Mavaddat 2015 | 1 | 10566215 | rs616488 |
| rs11552449 | Mavaddat 2015 | 1 | 114448389 | rs11552449 |
| rs11249433 | Mavaddat 2015 | 1 | 121280613 | rs11249433 |
| rs12405132 | Michailidou 2015 | 1 | 145644984 | rs12405132 |
| rs72755295 | Michailidou 2015 | 1 | 242034263 | rs72755295 |
| rs12710696 | Mavaddat 2015 | 2 | 19320803 | rs12710696 |
| rs4849887 | Mavaddat 2015 | 2 | 121245122 | rs4849887 |
| rs2016394 | Mavaddat 2015 | 2 | 172972971 | rs2016394 |
| rs1550623 | Mavaddat 2015 | 2 | 174212894 | rs1550623 |
| rs13387042 | Mavaddat 2015 | 2 | 217905832 | rs13387042 |
| rs16857609 | Mavaddat 2015 | 2 | 218296508 | rs16857609 |
| rs6762644 | Mavaddat 2015 | 3 | 4742276 | rs6762644 |
| rs4973768 | Mavaddat 2015 | 3 | 27416013 | rs4973768 |
| rs12493607 | Mavaddat 2015 | 3 | 30682939 | rs12493607 |
| rs6796502 | Michailidou 2015 | 3 | 46866866 | rs6796502 |
| rs1053338 | Michailidou 2015 | 3 | 63967900 | rs1053338 |
| rs9790517 | Mavaddat 2015 | 4 | 106084778 | rs9790517 |
| rs6828523 | Mavaddat 2015 | 4 | 175846426 | rs6828523 |
| rs10069690 | Mavaddat 2015 | 5 | 1279790 | rs10069690 |
| rs7726159 | Mavaddat 2015 | 5 | 1282319 | rs7726159 |
| rs2736108 | Mavaddat 2015 | 5 | 1297488 | rs2736108 |
| rs13162653 | Michailidou 2015 | 5 | 16187528 | rs13162653 |
| rs2012709 | Michailidou 2015 | 5 | 32567732 | rs2012709 |
| rs10941679 | Mavaddat 2015 | 5 | 44706498 | rs10941679 |
| rs889312 | Mavaddat 2015 | 5 | 56031884 | rs889312 |
| rs10472076 | Mavaddat 2015 | 5 | 58184061 | rs10472076 |
| rs1353747 | Mavaddat 2015 | 5 | 58337481 | rs1353747 |
| rs7707921 | Michailidou 2015 | 5 | 81538046 | rs7707921 |
| rs1432679 | Mavaddat 2015 | 5 | 158244083 | rs1432679 |
| rs11242675 | Mavaddat 2015 | 6 | 1318878 | rs11242675 |
| rs204247 | Mavaddat 2015 | 6 | 13722523 | rs204247 |
| rs17529111 | Mavaddat 2015 | 6 | 82128386 | rs17529111 |
| rs12662670 | Mavaddat 2015 | 6 | 151918856 | rs12662670 |
| rs2046210 | Mavaddat 2015 | 6 | 151948366 | rs2046210 |
| rs6964587 | Michailidou 2015 | 7 | 91630620 | rs6964587 |
| rs4593472 | Michailidou 2015 | 7 | 130667121 | rs4593472 |
| rs720475 | Mavaddat 2015 | 7 | 144074929 | rs720475 |
| rs9693444 | Mavaddat 2015 | 8 | 29509616 | rs9693444 |
| rs13365225 | Michailidou 2015 | 8 | 36858483 | rs13365225 |
| rs6472903 | Mavaddat 2015 | 8 | 76230301 | rs6472903 |
| rs2943559 | Mavaddat 2015 | 8 | 76417937 | rs2943559 |
| rs13267382 | Michailidou 2015 | 8 | 117209548 | rs13267382 |
| rs13281615 | Mavaddat 2015 | 8 | 128355618 | rs13281615 |
| rs11780156 | Mavaddat 2015 | 8 | 129194641 | rs11780156 |
| rs1011970 | Mavaddat 2015 | 9 | 22062134 | rs1011970 |
| rs10759243 | Mavaddat 2015 | 9 | 110306115 | rs10759243 |
| rs865686 | Mavaddat 2015 | 9 | 110888478 | rs865686 |
| rs7072776 | Mavaddat 2015 | 10 | 22032942 | rs7072776 |
| rs11814448 | Mavaddat 2015 | 10 | 22315843 | rs11814448 |
| rs10995190 | Mavaddat 2015 | 10 | 64278682 | rs10995190 |
| rs704010 | Mavaddat 2015 | 10 | 80841148 | rs704010 |
| rs7904519 | Mavaddat 2015 | 10 | 114773927 | rs7904519 |
| rs11199914 | Mavaddat 2015 | 10 | 123093901 | rs11199914 |
| rs2981579 | Mavaddat 2015 | 10 | 123337335 | rs2981579 |
| rs3817198 | Mavaddat 2015 | 11 | 1909006 | rs3817198 |
| rs3903072 | Mavaddat 2015 | 11 | 65583066 | rs3903072 |
| rs78540526 | Mavaddat 2015 | 11 | 69331418 | rs78540526 |
| rs554219 | Mavaddat 2015 | 11 | 69331642 | rs554219 |
| rs75915166 | Mavaddat 2015 | 11 | 69379161 | rs75915166 |
| rs11820646 | Mavaddat 2015 | 11 | 129461171 | rs11820646 |
| rs10771399 | Mavaddat 2015 | 12 | 28155080 | rs10771399 |
| rs17356907 | Mavaddat 2015 | 12 | 96027759 | rs17356907 |
| rs1292011 | Mavaddat 2015 | 12 | 115836522 | rs1292011 |
| rs11571833 | Mavaddat 2015 | 13 | 32972626 | rs11571833 |
| rs2236007 | Mavaddat 2015 | 14 | 37132769 | rs2236007 |
| rs2588809 | Mavaddat 2015 | 14 | 68660428 | rs2588809 |
| rs999737 | Mavaddat 2015 | 14 | 69034682 | rs999737 |
| rs941764 | Mavaddat 2015 | 14 | 91841069 | rs941764 |
| rs11627032 | Michailidou 2015 | 14 | 93104072 | rs11627032 |
| rs3803662 | Mavaddat 2015 | 16 | 52586341 | rs3803662 |
| rs17817449 | Mavaddat 2015 | 16 | 53813367 | rs17817449 |
| rs13329835 | Mavaddat 2015 | 16 | 80650805 | rs13329835 |
| chr17: 29239529: D | Michailidou 2015 | 17 | 29230520 | rs62070643** |
| rs6504950 | Mavaddat 2015 | 17 | 53056471 | rs6504950 |
| rs745570 | Michailidou 2015 | 17 | 77781725 | rs745570 |
| rs527616 | Mavaddat 2015 | 18 | 24337424 | rs527616 |
| rs1436904 | Mavaddat 2015 | 18 | 24570667 | rs1436904 |
| rs6507583 | Michailidou 2015 | 18 | 42399590 | rs6507583 |
| rs8170 | Mavaddat 2015 | 19 | 17389704 | rs8170 |
| rs2363956 | Mavaddat 2015 | 19 | 17394124 | rs2363956 |
| rs4808801 | Mavaddat 2015 | 19 | 18571141 | rs4808801 |
| rs3760982 | Mavaddat 2015 | 19 | 44286513 | rs3760982 |
| rs2823093 | Mavaddat 2015 | 21 | 16520832 | rs2823093 |
| rs17879961 | Mavaddat 2015 | 22 | 29121087 | rs17879961 |
| rs132390 | Mavaddat 2015 | 22 | 29621477 | rs132390 |
| rs6001930 | Mavaddat 2015 | 22 | 40876234 | rs6001930 |

* Originally published variant substituted by LD SNP, only SNPs with R2 ≥ 0.9 are listed.
**SNP in LD with published variant.

### Cancer Methods and Treatment

Cancer therapy can include surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound including, for example, a biologic or exogenous active agent.

Examples of treatments include bariatric surgical intervention, physical therapy, diet, and diet supplementation.

Examples of a cancer biological therapy include adoptive cell transfer, angiogenesis inhibitors, bacillus Calmette-Guerin therapy, biochemotherapy, cancer vaccines, chimeric antigen receptor (CAR) T-cell therapy, cytokine therapy, gene therapy, immune checkpoint modulators, immunoconjugates, monoclonal antibodies, oncolytic virus therapy, and targeted drug therapy.

Examples of a cancer surgery include lumpectomy, partial mastectomy, total mastectomy, simple mastectomy, modified radical mastectomy, radical mastectomy, and Halsted radical mastectomy.

Examples of a cancer drug include drugs approved to prevent breast cancer including Evista (Raloxifene Hydrochloride), Raloxifene Hydrochloride, and Tamoxifen Citrate.

Examples of a cancer drug include drugs approved to treat breast cancer including, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate).

As used herein, the term “disease” includes any disorder, condition, sickness, ailment that manifests in, for example, a disordered or incorrectly functioning organ, part, structure, or system of the body.

As used herein, the term “sample” includes any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including synovial fluid, gingival crevicular fluid, bone marrow, cerebrospinal fluid (CSF), saliva, mucous, sputum, semen, sweat, urine, or any other bodily fluids. A blood sample can include whole blood or any fraction thereof, including blood cells, red blood cells, white blood cells or leucocytes, platelets, serum and plasma.

As used herein, the term “subject” includes humans. Humans generally include women and men and others such as non-binary.

In some embodiments, this invention can provide methods for recommending therapeutic regimens, including withdrawal from therapeutic regiments.

In further embodiments, an odds ratio can provide a clinician with a prognostic picture of a subject's biological state. Such embodiments may provide subject-specific prognostic information, which can be informative for a therapy decision, and may also facilitate monitoring therapy response. Such embodiments may result in a surprisingly improved treatment, such as better control of a disease, or an increase in the proportion of subjects achieving amelioration of symptoms.

As used herein, the terms “biologic,” “biotherapy,” and/or “biopharmaceutical” can include pharmaceutical therapy products manufactured or extracted from a biological substance. A biologic can include vaccines, blood or blood components, allergenics, somatic cells, gene therapies, tissues, recombinant proteins, and living cells; and can be composed of sugars, proteins, nucleic acids, living cells or tissues, or combinations thereof.

As used herein, the terms “therapeutic regimen,” “therapy” and/or “treatment” can include any clinical management of a subject, as well as interventions, whether biological, chemical, physical, or a combination thereof, intended to sustain, ameliorate, improve, or otherwise alter the condition of a subject.

As used herein, the term “administering” can include the placement of a composition into a subject by a method or route that results in at least partial localization of the composition at a desired site such that a desired effect is produced. Routes of administration include both local and systemic administration. Generally, local administration results in more of the composition being delivered to a specific location as compared to the entire body of the subject, whereas, systemic administration results in delivery to essentially the entire body of the subject. “Administering” also includes performing physical actions on a subject's body, including physical therapy, as well as chiropractic care, massage and acupuncture.

### Devices and Systems

As used herein, the term machine-readable storage medium can comprise, for example, a data storage material that is encoded with machine-readable data or data arrays. The data and machine-readable storage medium may be capable of being used for a variety of purposes, when using a machine programmed with instructions for using said data. Such purposes include storing, accessing and manipulating information relating to the risk of a subject or population over time, or risk in response to treatment, or for drug discovery for inflammatory disease. Data comprising genomic measurements can be implemented in computer programs that are executing on programmable computers, which may comprise a processor, a data storage system, one or more input devices, one or more output devices. Program code can be applied to the input data to perform the functions described herein, and to generate output information. Output information can then be applied to one or more output devices. A computer can be, for example, a personal computer, a microcomputer, or a workstation.

As used herein, the term computer program can be instruction code implemented in a high-level procedural or object-oriented programming language, to communicate with a computer system. The program may be implemented in machine or assembly language. The programming language can also be a compiled or interpreted language. Each computer program can be stored on storage media or a device such as ROM, or magnetic diskette, and can be readable by a programmable computer for configuring and operating the computer when the storage media or device is read by the computer to perform the described procedures. A health-related or genomic data management system can be considered to be implemented as a computer-readable storage medium, configured with a computer program, where the storage medium causes a computer to operate in a specific manner to perform various functions.

### CONCLUSION

All publications, patents and literature specifically mentioned herein are hereby incorporated by reference in their entirety for all purposes.

A reference SNP ID number, or “rs” ID, is an identification tag assigned by NCBI to a group or cluster of SNPs that map to an identical location. The rs ID number, or rs tag, is assigned after submission. A submitted SNP is evaluated to see if it maps to an identical location as previously submitted SNPs; if it does, then the submitted SNP is linked into the reference set of the existing reference SNP record. These SNP rs IDs are mapped to external resources or databases, including NCBI databases. The SNP rs ID number is noted on the records of these external resources and databases in order to point users back to the original dbSNP records. A reference SNP record has the format NCBI| rs<NCBI SNP ID>.

Words specifically defined herein have the meaning provided in the context of the present disclosure as a whole, and as are typically understood by those skilled in the art. As used herein, the singular forms “a,” “an,” and “the” include the plural.

While the present disclosure is described in conjunction with various embodiments, it is not intended that the present disclosure be limited to such embodiments. On the contrary, the present disclosure encompasses various alternatives, modifications, and equivalents, as will be appreciated by those of skill in the art.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples herein are illustrative only and not intended to be limiting.

Although the foregoing disclosure has been described in some detail by way of illustration and examples for purposes of clarity of understanding, it will be understood by persons of skill in the art that various changes and modifications may be practiced within the scope of the invention and the appended claims.

### EXAMPLES

### Example 1: Global Polygenic Breast Cancer Risk Assessment

Breast cancer risk assessment was determined by using single-nucleotide polymorphisms (SNPs) with small effects that were aggregated into a global polygenic risk score (gPRS), primarily developed and validated for populations of European descent. To make a gPRS meaningful for all women of all heritage groups and populations, a novel global PRS (gPRS) was determined and validated that utilizes individual ancestral genetic composition.

Ancestry-specific global polygenic risk scores corresponding to three continental ancestries were determined and validated using 149 SNPs comprised of 93 breast cancer-associated SNPs and 56 ancestry-informative SNPs. An African polygenic risk score was determined using a cohort of 31,126 self-reported African American patients referred for hereditary cancer testing. An East Asian polygenic risk score was developed based on published data from the Asia Breast Cancer Consortium. A European polygenic risk score was determined using data from the Breast Cancer Association Consortium and 24,259 European hereditary cancer testing patients. For each patient, ancestry-informative SNPs were used to calculate the fractional ancestry attributable to each of the three continents. The gPRS was the sum of ancestry-specific polygenic risk scores weighted according to genetic ancestral composition. In an independent validation cohort (N=62,707), discrimination and calibration of the gPRS were evaluated and compared for performance against a previously described 86-SNP PRS for women of European ancestry. Associations of SNPs and polygenic risk scores with breast cancer were analyzed using logistic regression adjusted for personal and family cancer history, age, and ancestry. Odds ratios (ORs) were reported per standard deviation within the corresponding patient population. P-values were reported as two-sided.

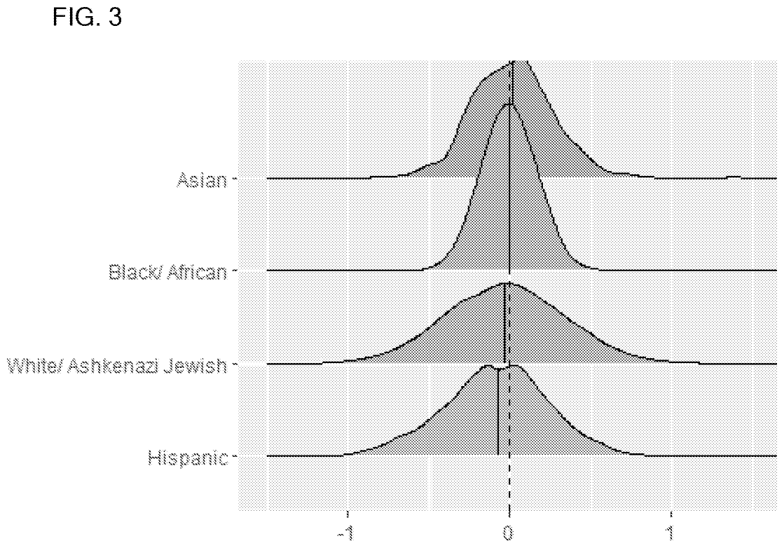

FIG. 3 shows an illustration of an embodiment of this invention in which the distribution of Global Polygenic Risk Scores for breast cancer risk was centered about zero for patients of all ancestry genotypes indicating removal of ancestry-derived bias from risk estimation.

As shown in Table 4, the gPRS was strongly associated with breast cancer in the full validation cohort and in sub-cohorts defined by self-reported ancestry.

TABLE 4
Global polygenic risk estimation for breast cancer

| Self-reported | | | |
|---|---|---|---|
| ancestry | N | OR (95% CI) | p-value |
| All | 62,707 | 1.41 (1.38-1.44) | 2.5 × 10⁻²¹² |
| Asian | 1,325 | 1.25 (1.07-1.45) | 3.7 × 10⁻³ |
| Black/African | 6,743 | 1.23 (1.16-1.31) | 8.5 × 10⁻¹¹ |
| Hispanic | 5,847 | 1.35 (1.24-1.46) | 1.6 × 10⁻¹³ |
| Mixed ancestry | 2,681 | 1.59 (1.39-1.82) | 2.4 × 10⁻¹² |
| Non-European | 14,959 | 1.29 (1.23-1.36) | 2.5 × 10⁻²⁵ |
| White and/or | 42,897 | 1.44 (1.40-1.48) | 6.3 × 10⁻¹⁷² |
| Ashkenazi | | | |

Referring to Table 4, 95% (88/93) of breast cancer SNPs had ≥1% frequency of risk alleles within each of the self-reported populations. Compared to the previously described 86-SNP PRS, the gPRS of this invention showed improved discrimination overall, and within each sub-cohort, with the exception of the Asian population where the sample size was too small to show superiority of either score. The gPRS was properly calibrated for all women.

In conclusion, the 149-SNP gPRS was validated and calibrated for women of all ancestries. Combined with clinical and biological risk factors, the 149-SNP gPRS can provide surprisingly improved risk stratification for all women, regardless of ancestry, population or heritage.

### Example 2: Global Polygenic Breast Cancer Risk Determination

A global polygenomic risk estimation for breast cancer in all genetic ancestries was defined using a combination of 93 breast cancer-associated SNPs comprised of 92 European breast cancer-associated SNPs and one Hispanic breast cancer-associated SNP 6q25 (rs140068132).

European SNP breast cancer risk betas were based on meta-analysis of known SNP properties and SNP properties derived from myRisk patient data. African SNP breast cancer risk betas were based on data from 31,126 myRisk patients of self-reported African ancestry. Asian SNP breast cancer risk betas for about 70 SNPs were based on Shu et al., Nat Commun., 2020, Vol. 11, pp. 1217-1226. European SNP breast cancer risk betas were directly determined. Hispanic SNP breast cancer risk betas were determined from data for about 9,000 Hispanic myRisk patients.

A global polygenomic risk estimation for breast cancer was determined by a primary analysis involving evaluation of discrimination of the global polygenic risk score in a large cohort of myRisk patients of all ancestries.

The global polygenomic risk estimation for breast cancer was further determined by a secondary analysis involving calculating improvement of breast cancer risk discrimination for all populations using the 93 breast cancer-associated SNPs as compared to using an 86-SNP PRS in the full cohort. The secondary analysis was repeated in sub-cohorts defined by self-reported ancestry.

The global polygenomic risk estimation for breast cancer was further determined by an additional analysis confirming that the global polygenomic risk score was centered at zero for unaffected patients overall, and for each subpopulation except Hispanic carriers of the 6q25 SNP (rs140068132). Being centered at zero showed that the global polygenomic risk score was not biased toward any particular heritage group or population. Being centered at zero showed that the global polygenomic risk score surprisingly provided the same risk estimation for all heritage groups and populations.

As shown in Table 5, the global polygenomic risk estimation for breast cancer was therefore validated to provide breast cancer risk discrimination for all patients, and breast cancer risk discrimination for all sub-cohorts defined by self-reported ancestry.

TABLE 5
Global polygenic risk estimation for breast cancer

| Self-reported | | OR per SD | |
|---|---|---|---|
| ancestry | N | (95% CI) | p-value |
| All | 62,707 | 1.41 (1.38-1.44) | 1.4 × 10⁻²¹¹ |
| Asian | 1,325 | 1.25 (1.07-1.45) | 3.8 × 10⁻³ |
| Black/African | 6,743 | 1.22 (1.14-1.30) | 8.0 × 10⁻¹⁰ |
| Hispanic | 5,847 | 1.35 (1.24-1.46) | 1.7 × 10⁻¹³ |
| Mixed ancestry | 2,681 | 1.59 (1.39-1.81) | 2.80 × 10⁻¹² |
| Non-European | 14,959 | 1.29 (1.22-1.35) | 1.50 × 10⁻²⁴ |
| White and/or | 41,821 | 1.44 (1.40-1.48) | 5.70 × 10⁻¹⁶⁸ |
| Ashkenazi | | | |

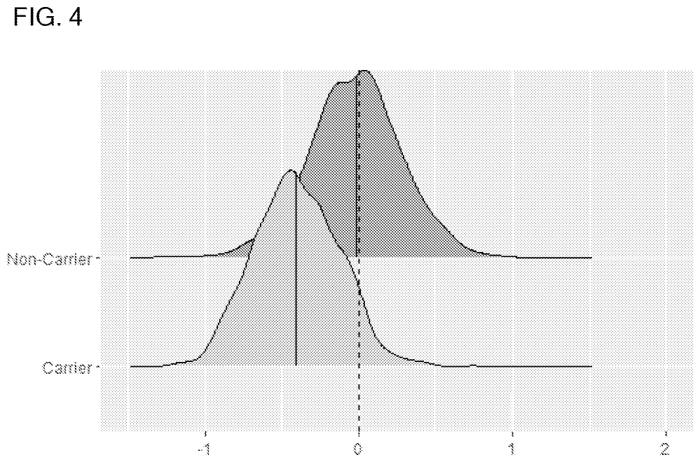

FIG. 4 shows an illustration of an embodiment of this invention in which the distribution of Global Polygenic Risk Scores for breast cancer risk was centered about zero for Hispanic patients who do not carry a 6q25 SNP (rs140068132).

As shown in FIG. 4, the global polygenic risk estimation for breast cancer was centered around zero for unaffected patients of all ancestries, except for carriers of the Hispanic 6q25 SNP (rs140068132). Carriers of the Hispanic 6q25 SNP (rs140068132) were handled separately to preserve the protective effect. The mean global polygenic risk estimations for breast cancer in FIG. 4 are shown in Table 6.

TABLE 6
Distribution of mean global polygenic risk estimations
(gPRS) for breast cancer by self-reported ancestry

| Self-reported | | |
|---|---|---|
| ancestry | N | Mean gPRS |
| Asian | 929 | 0.017 |
| Black/African | 4,989 | −0.006 |
| Hispanic | 4,781 | −0.088 |
| Mixed ancestry | 2,281 | −0.029 |
| Non-European | 11,524 | −0.038 |
| White and/or | 31,707 | −0.029 |
| Ashkenazi | | |

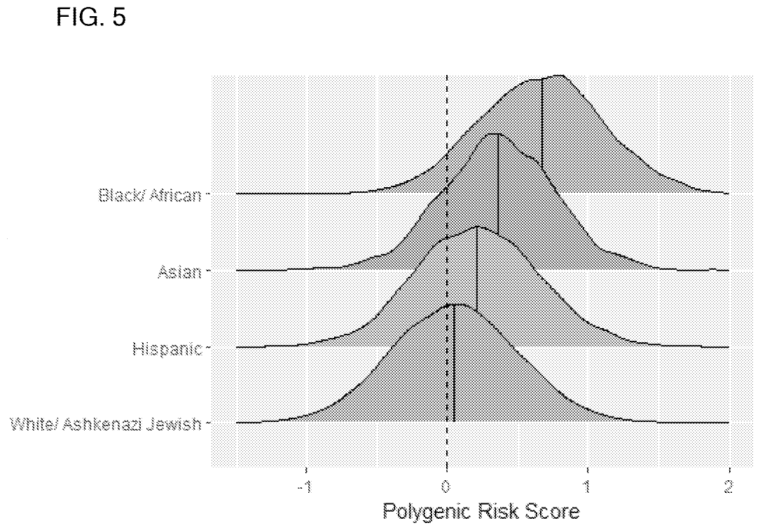

FIG. 5 shows a comparison of a ancestry-specific differences in distributions of polygenic risk scores based on different breast cancer allele frequencies.

As shown in FIG. 5, for Hispanic individuals, the global polygenic risk estimation for breast cancer was centered around zero for patients who do not carry the Hispanic 6q25 SNP (rs140068132), and was shifted toward lower risk for 6q25 (rs140068132) carriers.

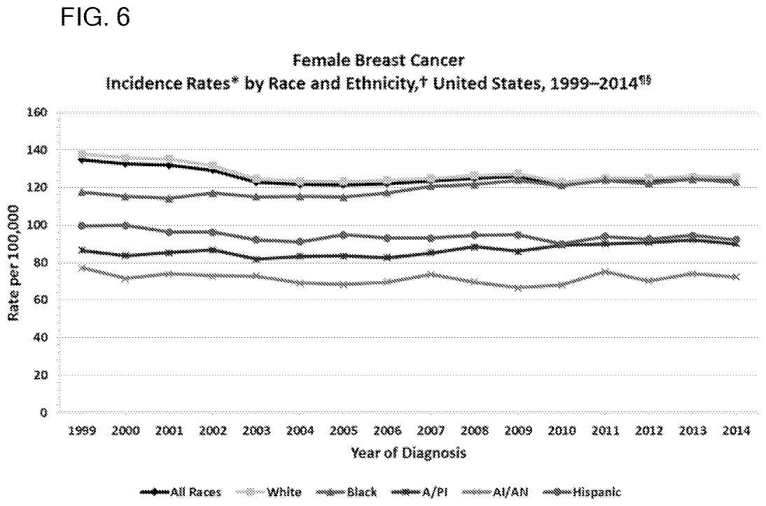

FIG. 6 shows an illustration of historical breast cancer rates by ancestry.

### Example 3: Use of a Comparative 86-SNP Polygenomic Risk Estimation

An 86-SNP polygenic risk score was evaluated separately for carriers of pathogenic variants in BRCA1, BRCA2, CHEK2, ATM, PALB2, and non-carriers. The use of an 86-SNP polygenomic risk estimation without other markers and elements was a comparative method.

An IRB-approved study included 152,012 women of European ancestry who were tested clinically for hereditary cancer risk with a multi-gene panel. An 86-SNP polygenic risk score was evaluated separately for carriers of pathogenic variants in BRCA1 (N=2,249), BRCA2 (N=2,638), CHEK2 (N=2,564), ATM (N=1,445) and PALB2 (N=906), and for non-carriers (N=141,160). Multivariable logistic regression was used to examine the association of the 86-SNP scores with invasive breast cancer after accounting for age and family cancer history. Effect sizes, expressed as standardized odds ratios (OR) with 95% confidence intervals (CIs), were assessed for carriers of each gene and for non-carriers. The 86-SNP score was strongly associated with breast cancer risk in BRCA1, BRCA2, CHEK2, ATM and PALB2 carrier populations (p<10-+). However, different effect sizes for different genes made further interpretation difficult.

The polygenic risk score was defined as a linear combination of centered risk alleles:

$$Polygenic Risk Score = {b}_{1} ( {x}_{1} - {u}_{1} ) + {b}_{2} ( {x}_{2} - {u}_{2} ) + … + {b}_{N} ( {x}_{N} - {u}_{N} )$$

where N was the total number of SNPs selected, the coefficient bₖ was the per-allele log OR for breast cancer association of the kth SNP estimated from meta-analysis of literature and the development cohort; xₖ was the number of alleles of the kth SNP carried by an individual patient (Xₖ=0, 1 or 2); and uₖ was the average number of alleles of the kth SNP reported for individuals included in large general population studies. Passing criteria restricted the number of missing SNP calls such that the imputation of missing calls by the high or low risk allele(s) did not change the relative risk by more than 10%.

Associations with invasive breast cancer were evaluated in terms of p-values and ORs with 95% confidence intervals (CI) from multivariate logistic regression models constructed using R version 3.4.4 or higher (R Foundation for Statistical Computing, Vienna, Austria). ORs were reported per unit standard deviation of the polygenic risk score (PRS) in unaffected controls. P-values were calculated from likelihood ratio chi-square test statistics and reported as two-sided. Using multivariable logistic regression addresses the implicit bias in a genetic testing cohort where patients are selected for a qualifying factor, BC diagnosis or family history. Adjustment for factors related to ascertainment in a clinical testing population may enable the derivation of unbiased risk estimates.

All models included independent variables for age of first invasive breast cancer (BC) diagnosis or age at genetic testing if unaffected, personal history of non-BC, family history of any cancer and ancestry, European and/or Ashkenazi Jewish. Cases were women diagnosed with invasive breast cancer, with or without ductal carcinoma in situ (DCIS). Controls were BC cancer free at time of testing. Women diagnosed with DCIS were excluded from controls. In testing for a relationship between PRS and age, the multivariate model included an interaction term for PRS and age. An interaction test was also performed for PRS and carrier status, testing for a difference in PRS performance by gene. In this model a categorical variable represented the carrier status, non-carrier, BRCA1 pathogenic variant, BRCA2 pathogenic variant, etc., the PRS was standardized within each carrier group and an interaction term for PRS and carrier status was included.

Models included clinical variables for age, personal cancer history, family cancer history, and ancestry. Data were derived from the test request form submitted for hereditary genetic testing. Since clinical variables were also used to define eligibility for the study cohort, only women with complete clinical data are included in the study.

Age was coded in years as a continuous variable. The age of first diagnosis of invasive breast cancer was used for affected patients and age at the time of genetic testing for unaffected patients. Personal cancer variables were coded as binary, ever or never affected. Separate variables were coded for uterine/endometrial cancer, ovarian cancer, pancreatic cancer, stomach cancer, non-polyposis colorectal cancer, and adenomatous polyposis patients with ≥20 polyps.

All patients were tested for germline mutations for the following genes: APC, ATM, BARD1, BMPR1A, BRCA1, BRCA2, BRIP1, CDH1, CDK4, CDKN2A (p14ARF, p16), CHEK2, EPCAM, MLH1, MSH2, MSH6, MYH, NBN, PALB2, PMS2, PTEN, RAD51C, RAD51D, SMAD4, STK11, and TP53. Library preparation encompassed custom designed targeted next-generation sequencing (NGS) reagents for both exonic segments and additional DNA segments carrying informative breast cancer (BC) single nucleotide polymorphisms (SNPs). Long-range and nested PCR were applied to portions of the CHEK2 gene to exclude pseudogene sequences. Sequencing on HiSeq2500 or MiSeq instruments (Illumina Inc., San Diego, CA) identified both sequence variants and large rearrangements (deletions and duplications).

The primary analysis examined the association of the 86-SNP score with invasive BC in each gene carrier group. In exploratory analyses the performance of the 86-SNP score in carriers of CHEK2 1100delC or other CHEK2 PVs were compared. To test for the interaction with family history, either a binary variable (presence or absence of an affected first-degree relative) or the sum of relatives affected with invasive BC in a weighted relative count was used. To test for interaction with gene carrier status a categorical variable for non-carrier or gene-specific carrier status was created.

Familial cancers were coded as numeric counts of diagnoses, weighted according to degree of relatedness. A weight of 0.5 was used for each first-degree relative and 0.25 for each second-degree relative. Variables included ductal invasive breast cancer, lobular invasive breast cancer (LCIS), DCIS, male breast cancer, prostate cancer, and each of the personal cancer types listed above. Ancestries were coded as quantitative variables representing fractions of reported ancestries. For example, a patient who listed only Ashkenazi ancestry was coded with an Ashkenazi value of 1.0, and zero for European ancestries. A patient who reported European and Ashkenazi ancestries was coded with European and Ashkenazi values of 0.5.

To examine relative risks by percentiles of the 86-SNP score, the non-carrier and BRCA1, BRCA2, CHEK2, and ATM PV-positive cohorts were each binned into quintiles based on the 86-SNP score. The PALB2 cohort was binned into tertiles to account for the smaller sample size. The median percentile bin (33rd-66th percentile tertile for PALB2, 40-60th percentile quintile for all others) was set as the reference group in a model that also included the above described covariates.

Absolute lifetime risks of developing BC were calculated for unaffected study participants by combining the 86-SNP score-based risk with previously-published gene-specific risk estimates (for PV carriers) or lifetime BC risk estimates from Surveillance, Epidemiology, and End Results (SEER) 2009-2014 data (for non-carriers).

Lifetime breast cancer risk as probability density function against absolute risk estimates by age 80 for carriers of pathogenic variants (PV) in breast cancer associated genes can be determined as modified by an 86-SNP score method. For women with pathogenic variants (PV) in moderate-risk breast cancer genes CHEK2, ATM, and PALB2, point estimates were higher than for BRCA1/2 carriers. The interaction between the 86-SNP score and gene carrier type was significant. The most pronounced risk discrimination was observed for CHEK2 carriers, where the effect size was equivalent to the odds ratios observed in non-carriers and for the general population.

A summary of the clinical characteristics and demographic data of the study cohort is shown in Table 7.

TABLE 7
Summary of the clinical characteristics
and demographic data of the study cohort

| Gene | Number of Women with a PV^{a} |
|---|---|
| ATM | 1,445 |
| BARD1 | 331 |
| BRCA1 | 2,249 |
| BRCA2 | 2,638 |
| CDH1 | 92 |
| CHEK2 | 2,564 |
| NBN | 440 |
| PALB2 | 906 |
| PTEN | 49 |
| STK11 | 7 |
| TP53 | 131 |

^{a}Subjects with more that one PV were excluded from the 86-SNP score risk modification analysis.

ORs for developing breast cancer for the continuous 86-SNP score in carriers of CHEK2 1100delC and other CHEK2 PVs is shown in Table 8.

TABLE 8
ORs for developing breast cancer for the continuous 86-SNP
score in carriers of CHEK2 1100delC and other CHEK2 PVs

| CHEK2 PV type | N | OR (95% CI) | p-value |
|---|---|---|---|
| 1100delC | 1426 | 1.38 (1.22-1.56) | 1.9 × 10⁻⁰⁷ |
| Other PV | 1138 | 1.67 (1.46-1.92) | 3.9 × 10⁻¹⁴ |

ORs for developing breast cancer for the continuous 86-SNP score by age bin and by carrier status for a PV in a BC-associated gene is shown in Table 9.

TABLE 9
ORs for developing breast cancer for the continuous 86-SNP score
by age bin and by carrier status for a PV in a BC-associated gene

| Pathogenic | | | | |
|---|---|---|---|---|
| Variant | Age (years) | N | OR (95% CI) | p-value ^{a} |
| None | <40 | 44,350 | 1.43 (1.38-1.49) | 1.2 × 10⁻⁸³ |
| (non-carriers) | ≥40-<49 | 39,227 | 1.52 (1.48-1.56) | 1.8 × 10⁻²³⁷ |
| | ≥50-<59 | 33,343 | 1.45 (1.41-1.49) | 4.6 × 10⁻¹⁶⁶ |
| | ≥60 | 24,240 | 1.44 (1.40-1.49) | 4.6 × 10⁻¹³⁰ |
| ATM | <40 | 448 | 1.27 (0.96-1.70) | 0.09 |
| | ≥40-<49 | 431 | 1.46 (1.17-1.82) | 5.9 × 10⁻⁴ |
| | ≥50-<59 | 329 | 1.34 (1.05-1.72) | 0.02 |
| | ≥60 | 237 | 1.67 (1.18-2.43) | 0.004 |
| BRCA1 | <40 | 1,086 | 1.22 (1.05-1.42) | 0.01 |
| | ≥40-<49 | 567 | 1.30 (1.09-1.55) | 0.004 |
| | ≥50-<59 | 371 | 1.20 (0.96-1.51) | 0.11 |
| | ≥60 | 225 | 1.02 (0.73-1.42) | 0.92 |
| BRCA2 | <40 | 992 | 1.19 (0.99-1.42) | 0.06 |
| | ≥40-<49 | 693 | 1.23 (1.05-1.45) | 0.009 |
| | ≥50-<59 | 529 | 1.20 (0.99-1.45) | 0.06 |
| | ≥60 | 424 | 1.19 (0.96-1.49) | 0.11 |
| CHEK2 | <40 | 817 | 1.25 (1.01-1.54) | 0.04 |
| | ≥40-<49 | 753 | 1.70 (1.44-2.01) | 1.2 × 10⁻⁹ |
| | ≥50-<59 | 607 | 1.44 (1.21-1.72) | 2.6 × 10⁻⁵ |
| | ≥60 | 387 | 1.42 (1.12-1.82) | 0.004 |
| PALB2 | <40 | 255 | 1.36 (0.97-1.93) | 0.08 |
| | ≥40-<49 | 271 | 1.46 (1.13-1.91) | 0.004 |
| | ≥50-<59 | 217 | 1.30 (0.96-1.77) | 0.10 |
| | ≥60 | 163 | 1.37 (0.94-2.04) | 0.10 |

^{a} p-value tests whether the OR is significantly different from 1.

ORs for developing breast cancer by BC affected status of a first-degree relative and by carrier status for a PV in a BC-associated gene is shown in Table 10.

TABLE 10
ORs for developing breast cancer by BC affected status of a
first-degree relative and by carrier status for a PV in a
BC-associated gene

| | First-Degree | | | First-Degree | | |
|---|---|---|---|---|---|---|
| | Relative without | | | Relative with | | |
| | Breast Cancer | | | Breast Cancer | | |
| PV Gene | N | OR | 95% CI | N | OR | 95% CI |
| None (non- | 92,529 | 1.47 | (1.44-1.49) | 48,631 | 1.48 | (1.44-1.51) |
| carriers) | | | | | | |
| BRCA1 | 1,308 | 1.18 | (1.05-1.34) | 941 | 1.25 | (1.09-1.44) |
| BRCA2 | 1,493 | 1.20 | (1.06-1.36) | 1,145 | 1.27 | (1.12-1.46) |
| CHEK2 | 1,436 | 1.65 | (1.45-1.89) | 1,128 | 1.34 | (1.18-1.53) |
| ATM | 809 | 1.27 | (1.07-1.52) | 636 | 1.44 | (1.21-1.73) |
| PALB2 | 461 | 1.27 | (1.01-1.59) | 445 | 1.34 | (1.10-1.65) |

A summary of the clinical characteristics and demographic data of the study cohort is shown in Table 11.

TABLE 11
Summary of the clinical characteristics and demographic data of the study cohort

| | | BRCA1 | BRCA2 | CHEK2 | ATM | PALB2 |
|---|---|---|---|---|---|---|
| | Non- | PV | PV | PV | PV | PV |
| Variable | Carriers | Carriers | Carriers | Carriers | Carriers | Carriers |
| Total Patients | 141,160 | 2,249 | 2,638 | 2,564 | 1,445 | 906 |
| Age at Hereditary Cancer | 48 (18, 84) | 4 (18, 84) | 47 (18, 84) | 48 (18, 84) | 49 (18, 84) | 51 (18, 82) |
| Testing, Median (Range) | | | | | | |

BC History

| | | | | | | |
|---|---|---|---|---|---|---|
| Personal BC, N (%) | 28,928 (20) | 828 (37) | 897 (34) | 914 (36) | 486 (34) | 401 (44) |
| ≥1 First-or Second- | 100,216 (71) | 1,700 (76) | 2,003 (76) | 1,972 (77) | 1,101 (76) | 720 (79) |
| Degree Relative, N (%) | | | | | | |

Ancestry

| | | | | | | |
|---|---|---|---|---|---|---|
| Ashkenazi Jewish, N (%) | 2,924 (2) | 69 (3) | 59 (2) | 24 (1) | 16 (1) | 8 (1) |
| White/Non-Hispanic, | 134,819 (96) | 2,115 (94) | 2,504 (95) | 2,504 (98) | 1,404 (97) | 886 (98) |
| N (%) | | | | | | |
| Ashkenazi Jewish and | 3,417 (2) | 65 (3) | 75 (3) | 36 (1) | 25 (2) | 12 (1) |
| White/Non-Hispanic, N | | | | | | |
| (%) | | | | | | |

Modification of risk of development of breast cancer by an 86-SNP polygenic risk score in carriers of a pathogenic variant in five BC-associated genes is shown in Table 12.

TABLE 12
Risk of breast cancer for 86-SNP polygenic
risk score in carriers of a PV

| PV Cohort | N | OR | 95% CI | p-value |
|---|---|---|---|---|
| ATM | 1,445 | 1.37 | 1.21-1.55 | 2.6 × 10⁻⁷ |
| BRCA1 | 2,249 | 1.20 | 1.10-1.32 | 6.5 × 10⁻⁵ |
| BRCA2 | 2,638 | 1.23 | 1.12-1.34 | 4.2 × 10⁻⁶ |
| PALB2 | 906 | 1.34 | 1.16-1.55 | 6.2 × 10⁻⁵ |
| CHEK2 | 2,564 | 1.49 | 1.36-1.64 | 1.3 × 10⁻¹⁸ |
| Non-carriers | 141,160 | 1.47 | 1.45-1.49 | <5 × 10⁻³²⁴ |

Odds ratios for developing breast cancer by percentile of an 86-SNP PRS and by carrier status for a pathogenic variant in a BC associated gene is shown in Tables 13 and 14.

TABLE 13
Risk of breast cancer for 86-SNP polygenic risk score in carriers of a PV

| 86-SNP | Non-Carriers | | ATM | | CHEK2 | |
|---|---|---|---|---|---|---|
| Score | OR | | OR | | OR | |
| Percentile | (95% CI) | p-value | (95% CI) | p-value | (95% CI) | p-value |
| ≤20 | 0.61 | 8.6 × 10⁻⁹⁰ | 0.46 | 1.7 × 10⁻⁴ | 0.59 | 5.6 × 10⁻⁴ |
| | (0.58-0.64) | | (0.31-0.69) | | (0.44-0.80) | |
| >20-≤40 | 0.85 | 3.4 × 10⁻¹² | 0.80 | 0.25 | 0.73 | 0.03 |
| | (0.81-0.89) | | (0.55-1.17) | | (0.54-0.97) | |
| >40-≤60^{a} | 1 | — | 1 | — | 1 | — |
| >60-≤80 | 1.30 | 6.4 × 10⁻³² | 1.25 | 0.23 | 1.42 | 0.01 |
| | (1.24-1.36) | | (0.87-1.80) | | (1.08-1.88) | |
| >80 | 1.79 | 1.5 × 10⁻¹⁶¹ | 1.18 | 0.38 | 1.67 | 3.0 × 10⁻⁴ |
| | (1.72-1.87) | | (0.82-1.71) | | (1.26-2.20) | |
| 86-SNP | PALB2 | | | | | |
| Score | OR | OR | | | | |
| Tertile | (95% CI) | (95% CI) | | | | |
| ≤33 | 0.68 | 0.04 | | | | |
| | (0.47-0.98) | | | | | |
| >33-≤66^{a} | 1 | — | | | | |
| >66 | 1.37 | 0.09 | | | | |
| | (0.96-1.95) | | | | | |

^{a}The middle percentile was used as the referent; p-values are for the difference in effect size between the percentile of the 86-SNP score and the referent group.

TABLE 14
Risk of breast cancer for 86-SNP polygenic risk score in carriers of a PV

| 86-SNP | Non-Carriers | | BRCA1 | | BRCA2 | |
|---|---|---|---|---|---|---|
| Score | OR | | OR | | OR | |
| Percentile | (95% CI) | p-value | (95% CI) | p-value | (95% CI) | p-value |
| ≤20 | 0.61 | 8.6 × 10⁻⁹⁰ | 0.82 | 0.18 | 0.67 | 0.006 |
| | (0.58-0.64) | | (0.61-1.10) | | (0.50-0.89) | |
| >20-≤40 | 0.85 | 3.4 × 10⁻¹² | 0.94 | 0.70 | 1.02 | 0.86 |
| | (0.81-0.89) | | (0.70-1.26) | | (0.78-1.35) | |
| >40-≤60^{a} | 1 | — | 1 | — | 1 | — |
| >60-≤80 | 1.30 | 6.4 × 10⁻³² | 1.08 | 0.59 | 1.11 | 0.44 |
| | (1.24-1.36) | | (0.81-1.45) | | (0.85-1.46) | |
| >80 | 1.79 | 1.5 × 10⁻¹⁶¹ | 1.52 | 0.004 | 1.31 | 0.054 |
| | (1.72-1.87) | | (1.14-2.03) | | (1.00-1.72) | |
| 86-SNP | PALB2 | | | | | |
| Score | OR | OR | | | | |
| Tertile | (95% CI) | (95% CI) | | | | |
| ≤33 | 0.68 | 0.04 | | | | |
| | (0.47-0.98) | | | | | |
| >33-≤66^{a} | 1 | — | | | | |
| >66 | 1.37 | 0.09 | | | | |
| | (0.96-1.95) | | | | | |

^{a}The middle percentile was used as the referent; p-values are for the difference in effect size between the percentile of the 86-SNP score and the referent group.

Estimated lifetime breast cancer risk to age 80 and modification by an 86-SNP PRS is shown in Table 15.

TABLE 15
Estimated lifetime breast cancer risk to age 80 and modification
by an 86-SNP PRS

| | Gene-Based | Adjusted Lifetime Risk | | | | |
|---|---|---|---|---|---|---|
| | Risk | Min | Q1 | Median | Q3 | Max |
| Gene | (%) | (%) | (%) | (%) | (%) | (%) |
| ATM | 28.2 | 12.9 | 23.9 | 29.0 | 34.7 | 58.3 |
| BRCA1 | 73.5 | 53.1 | 69.4 | 73.8 | 77.9 | 91.5 |
| BRCA2 | 73.8 | 50.8 | 69.0 | 74.2 | 78.9 | 94.2 |
| CHEK2 | 22.1 | 6.6 | 18.1 | 23.0 | 29.1 | 70.6 |
| PALB2 | 50.1 | 26.2 | 44.4 | 50.3 | 57.3 | 79.2 |
| Non-carriers | 12.7 | 2.5 | 10.4 | 13.2 | 16.9 | 62.4 |

What is claimed is:

1. A method for assessing ancestry of a subject, the method comprising:
selecting a plurality of ancestry-informative SNP markers based on the criteria:
the SNP markers substantially cover the entirety of the human genome;
the SNP markers each have at least 1% genomic frequency; and
the SNP markers have different frequencies in different heritage populations;
measuring a genotype of the subject; and
calculating a fractional heritage in the genotype of the subject for each of the different heritage populations based on the plurality of ancestry-informative SNP markers.

2. The method of claim 1, wherein the ancestry-informative SNP markers have different frequencies in three or more different heritage populations.

3. The method of claim 1, wherein the ancestry-informative SNP markers have different frequencies in African, European, and East Asian heritage populations.

4. The method of claim 1, wherein the plurality of ancestry-informative SNP markers is from 10 to 50,000 SNP markers.

5. The method of claim 1, wherein the plurality of ancestry-informative SNP markers is from 10 to 56 SNP markers.

6. A method for assessing a risk of a trait in a subject, the method comprising:
selecting a plurality of ancestry-informative SNP markers based on the criteria:
the SNP markers substantially cover the entirety of the human genome;
the SNP markers each have at least 1% genomic frequency; and
the SNP markers have different frequencies in different heritage populations;
measuring a genotype of the subject;
obtaining trait-associated SNP markers; and
calculating a global polygenic risk score for the risk of the trait in the subject based on the plurality of ancestry-informative SNP markers and the trait-associated SNP markers.

7. The method of claim 6, further comprising calculating the global polygenic risk score for the risk of the trait in the subject with additional clinical variables of the subject.

8. The method of claim 7, wherein the additional clinical variables are age, personal medical history, and family medical history of the subject.

9. The method of claim 6, wherein the trait is a risk of a disease in the subject.

10. The method of claim 9, wherein the disease is cancer.

11. The method of claim 6, wherein the plurality of ancestry-informative SNP markers are from 10 to 50,000 SNP markers.

12. The method of claim 6, wherein the plurality of ancestry-informative SNP markers are from 10 to 56 SNP markers.

13. The method of claim 6, wherein the trait-associated SNP markers are a plurality of cancer-associated SNP markers.

14. The method of claim 6, wherein the trait-associated SNP markers are a plurality of from 10 to 50,000 breast cancer-associated SNP markers.

15. The method of claim 6, wherein the trait-associated SNP markers are a plurality of from 10 to 93 breast cancer-associated SNP markers.

16. The method of claim 6, wherein the calculating a global polygenic risk score for the risk of the trait in the subject is done with training clinical data of a reference group.

17. The method of claim 6, wherein the calculating a global polygenic risk score for the risk of the trait in the subject is done with validating clinical data of a reference group.

18. The method of claim 6, wherein the genotype of the subject is measured by NGS.

19. The method of claim 6, wherein the genotype of the subject is determined with a sequencing chip.

20. The method of claim 6, wherein the plurality of ancestry-informative SNP markers determine a fractional heritage in the genotype of the subject for each of three or more different heritage populations.

21. The method of claim 6, wherein the plurality of ancestry-informative SNP markers determine a fractional heritage in the genotype of the subject for each of African, European, and East Asian heritage populations.

22. The method of claim 6, wherein the global polygenic risk score for the risk of the trait in the subject is accurate for subjects in three or more different heritage populations, even when the heritage populations are self-reported.

23. The method of claim 6, wherein the global polygenic risk score for the risk of the trait in the subject is accurate for subjects in African, European, and East Asian heritage populations, even when the heritage populations are self-reported.

24. The method of claim 6, wherein the global polygenic risk score for the risk of the trait in the subject is calibrated for subjects in three or more different heritage populations so that the risk of the trait is not overestimated in any heritage population.

25. The method of claim 6, wherein the global polygenic risk score for the risk of the trait in the subject is calibrated for subjects in African, European, and East Asian heritage populations so that the risk of the trait is not overestimated in any heritage population.

26. The method of claim 6, wherein the global polygenic risk score for the risk of the trait in the subject discriminates between low risk and high risk for subjects in three or more different heritage populations.

27. The method of claim 6, wherein the global polygenic risk score for the risk of the trait in the subject discriminates between low risk and high risk for subjects in African, European, and East Asian heritage populations.

28. The method of any one of claims 22-27, wherein the trait is a risk of a disease in the subject.

29. The method of claim 28, wherein the disease is cancer.

30. The method of claim 6, wherein the calculating a global polygenic risk score comprises using clinical cohorts of women of African self-reported ancestry, East Asian self-reported ancestry, and European self-reported ancestry.

31. The method of claim 6, wherein the calculating a global polygenic risk score comprises using the sum of ancestry specific polygenic risk scores weighted according to fractional ancestral composition.

32. The method of claim 6, wherein the global polygenic risk score is strongly associated with breast cancer in a reference cohort and in sub-cohorts defined by self-reported ancestry, 33 The method of claim 6, wherein the global polygenic risk score is combined with clinical and/or biological risk factors for accurate risk stratification for all women of all ancestries.

34. The method of claim 6, wherein the calculating a global polygenic risk score comprises a linear combination of risk alleles according to Equation III,
Polygenic Risk Score=b₁(x₁−u₁)+b₂(x₂−u₂)+ . . . +b_{N}(X_{N}−U_{N}) Equation III;
where N is the total number of SNPs selected;
the coefficient bₓ is the per-allele log OR for trait association of the kth SNP estimated from a development cohort;
Xₖ is the number of alleles of the kth SNP carried by an individual patient which is 0, 1 or 2;
and uₖ is the average number of alleles of the kth SNP reported for individuals included in large general population studies.

35. A method for treating a disease in a subject in need thereof, the method comprising:
selecting a plurality of ancestry-informative SNP markers based on the criteria:
the SNP markers substantially cover the entirety of the human genome;
the SNP markers each have at least 1% genomic frequency; and
the SNP markers have different frequencies in different heritage populations;
measuring a genotype of the subject;
obtaining disease-associated SNP markers; and
calculating a global polygenic risk score for the risk of the disease in the subject based on the plurality of ancestry-informative SNP markers and the disease-associated SNP markers, wherein the score indicates a need for treating the subject; and
administering to the subject a therapy for the disease.

36. The method of claim 35, further comprising calculating the global polygenic risk score with additional variables for age, personal medical history, and family medical history.

37. The method of claim 35, wherein the disease is cancer.

38. The method of claim 37, wherein the therapy is a cancer therapy selected from one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

39. The method of claim 37, wherein the disease is breast cancer.

40. The method of claim 39, wherein the therapy is a breast cancer therapy.

41. A method for diagnosing or prognosing a subject having a disease, the method comprising:
selecting a plurality of ancestry-informative SNP markers based on the criteria:
the SNP markers substantially cover the entirety of the human genome;
the SNP markers each have at least 1% genomic frequency; and
the SNP markers have different frequencies in different heritage populations;
measuring a genotype of the subject;
obtaining disease-associated SNP markers; and
calculating a global polygenic risk score for the risk of the disease in the subject based on the plurality of ancestry-informative SNP markers and the disease-associated SNP markers, wherein the score indicates a diagnosis or prognosis for the subject.

42. The method of claim 41, wherein the disease is cancer.

43. A method for generating data for assessing a trait in a subject, the method comprising:
selecting a plurality of ancestry-informative SNP markers based on the criteria:
the SNP markers substantially cover the entirety of the human genome;
the SNP markers each have at least 1% genomic frequency; and
the SNP markers have different frequencies in different heritage populations;
measuring a genotype of the subject;
measuring trait-associated SNP markers in the genotype of the subject.

44. The method of claim 43, further comprising determining additional clinical variables of the subject.

45. The method of claim 44, wherein the additional clinical variables are age, personal medical history, and family medical history of the subject, 46 The method of claim 43, wherein the trait is a risk of a disease in the subject.

47. The method of claim 46, wherein the disease is cancer.

48. The method of claim 43, wherein the plurality of ancestry-informative SNP markers are from 10 to 50,000 SNP markers.

49. The method of claim 43, wherein the plurality of ancestry-informative SNP markers are from 10 to 56 SNP markers.

50. The method of claim 43, wherein the trait-associated SNP markers are a plurality of cancer-associated SNP markers.

51. The method of claim 43, wherein the trait-associated SNP markers are a plurality of from 10 to 50,000 breast cancer associated SNP markers.

52. The method of claim 43, wherein the trait-associated SNP markers are a plurality of from 10 to 93 breast cancer associated SNP markers.

53. A system for assessing risk of a disease in a subject, the system comprising:
a processor for receiving a genotype of the subject;
one or more processors for carrying out the steps:
calculating a global polygenic risk score for risk of the disease in the subject based on a plurality of ancestry-informative SNP markers, a plurality of disease-associated SNP markers of the genotype, and additional variables for age, personal medical history, and family medical history; and
a display for displaying and/or reporting the risk score.

54. The system of claim 53, wherein the disease is cancer.

55. A non-transitory machine-readable storage medium having stored therein instructions for execution by a processor which cause the processor to perform the steps of a method for assessing risk of a disease in a subject, the method comprising:
receiving a genotype of the subject;
calculating a global polygenic risk score for risk of the disease in the subject based on a plurality of ancestry-informative SNP markers, a plurality of disease-associated SNP markers of the genotype, and additional variables for age, personal medical history, and family medical history; and
sending to a processor output for displaying and/or reporting the risk score.

56. The medium of claim 55, wherein the disease is cancer.
